(19) United States
(12) Patent Application Publication
(10) Pub. No.: US 2015/0209264 A1
(43) Pub. Date: Jul. 30, 2015

(54) COSMETIC OR PHARMACEUTICAL MOISTURISING INGREDIENT

(71) Applicant: BASF Beauty Care Solutions France S.A.S., Lyon (FR)

(72) Inventors: Isabelle Bonnet, Lyon (FR); Nicolas Verrecchia, Chassieu (FR)

(21) Appl. No.: 14/421,308

(22) PCT Filed: Aug. 13, 2013
(86) PCT No.: PCT/FR2013/051933

§ 371 Date: Feb. 12, 2015

Related U.S. Application Data
(60) Provisional application No. 61/684,983, filed on Aug. 20, 2012.

(30) Foreign Application Priority Data
Aug. 13, 2012 (FR) 1257786

(51) Int. Cl. A61K 8/73; A61K 8/60; A61K 8/44; A61K 8/42; A61K 31/719; A61Q 19/10; A61Q 19/08; A61K 31/734; A61K 31/728; A61Q 19/00; A61Q 1/00

(52) U.S. Cl. CPC A61K 8/735; A61Q 19/00; A61K 8/732; A61K 8/733; A61K 8/60; A61K 8/44; A61K 8/42; A61Q 1/00; A61Q 19/10; A61Q 19/08; A61K 31/734; A61K 31/728; A61K 31/719; A61K 2800/5922

(57) ABSTRACT

The present invention relates to the combination of pullulan or a derivative thereof with a polysaccharide chosen from hyaluronic acid, a salt or derivative thereof, alginic acid, a salt or derivative thereof and a mixture of these polysaccharides. It also relates to a mixture of cosmetic or pharmaceutical ingredients, a cosmetic or pharmaceutical composition and a detergent or hygiene product comprising this combination. It further relates to the use of this combination for reducing the insensible water losses and/or for increasing the water content of the skin and/or mucous membranes and/or for increasing and/or prolonging the content of cosmetic and/or pharmaceutical active ingredients in the skin and/or the mucous membranes and/or maintaining it over time, the use of this cosmetic composition for maintaining or improving the state of hydration of the skin, and/or for preventing or slowing down the appearance of the signs of cutaneous and/or mucosal dryness, and/or for treating cutaneous dryness conditions, and/or for preventing or reducing the appearance of wrinkles linked to cutaneous dryness, and/or for improving the comfort of dry skin and/or mucous membranes, and/or for treating skin and/or mucous membranes having an appearance that is rough to look at and/or to the touch, and also the use of the pharmaceutical composition in the treatment and/or the prevention of fissures and/or scurf or pityriasis alba and/or cracks and/or atopic dermatitis and/or ichthyosis and/or conditions of dryness of the skin or mucous membrane that accompany cutaneous pathologies.

The present invention relates to the cosmetic field and to the pharmaceutical, in particular dermatological, field. The present invention relates very particularly to a novel mixture of ingredients that makes it possible to reduce the insensible water losses and thus maintain the hydration of the skin.

The skin is an organ that is in constant contact with the outside. It therefore plays an exchange role but also, and above all, a barrier role with respect to the outside.

The surface layer of the skin, known as the horny layer, in effect constitutes the first barrier protecting the skin, in the form of a fine film. This layer, due to its hydrophobic nature, constitutes a barrier to the diffusion of water, the main constituent of the cells, thus preventing dehydration. Nevertheless, there are still losses of water, known as transepidermal water losses or otherwise known as insensible water losses. If the horny layer is weakened, in particular by aggressions or in pathological situations, the cutaneous barrier is broken and these insensible water losses increase. The skin becomes dry and more vulnerable to outside aggressions. If this cutaneous dehydration continues, it may induce actual pathologies. The maintaining of the water content via the cutaneous barrier role is therefore essential for maintaining the functionalities and the aesthetic appearance of the skin.

The provision of moisturizing active agents and the limitation of the insensible water losses are the two main axes for maintaining and strengthening this cutaneous barrier and consequently the good general condition of the skin. Novel moisturizing active agents and/or active agents capable of reducing the insensible water losses therefore are in this way of great interest on a cosmetic and pharmaceutical, in particular dermatological, level.

To date, many moisturizing active agents exist, such as those described in patent applications WO2005/095462 and FR2902333. However, the existing compositions currently on the market have only a short duration moisturizing power. There is therefore the need to be able to maintain the hydration of the skin over a long duration, that is to say over at least 10 hours, or even 24 hours.

Pullulan is a natural polysaccharide (saccharide polymer) constituted of maltotriose glucose trisaccharide) units, also known as α-1,4-; α-1,6-glucan. The three glucose units that make up the maltotriose are connected by an α-1,4 glycosidic bond, whereas the maltotrioses are connected to each other by an α-1,61 glycosidic bond. Pullulan has multiple applications.

It is above all known as being a thickener or film-forming agent as described in patent application US 2003/0082221. It is also known for its ability to capture and retain water as described in patent application FR7434619.

Hyaluronic acid, the salts and derivatives thereof are among the moisturizing active agents that are the best known and the most used at the present time. Hyaluronic acid is a polymer of disaccharides, themselves composed of D-glucuronic acid and D-N-acetylglucosamine, bonded to one another by alternate β-4,4 and β-1,3 glycosidic bonds. It is therefore a natural glycosaminoglycan. This is one of the main components of the extracellular matrix. Its various fractions, as a function of their molecular weights can be used as a moisturizing agent as described in patent application US 2008/0003271.

The stabilization of hyaluronic acid was investigated in patent application US 2009/0042834 which describes the combination of a glycosaminoglycan composition such as hyaluronic acid with a stabilizing agent chosen from long chain hydroxy polyanionic polysaccharides such as sodium alginate and alginic acid. The objective of this stabilization is to overcome the loss of viscosity observed with time in hyaluronic acid formulations. Such hyaluronic acid compositions stabilized in this way are used for the customary uses of hyaluronic acid, in particular its moisturizing properties. No improvement of the properties of the hyaluronic acid is however described, with the exception of a better maintaining of the properties thereof in formulation. Moreover, it is not suggested to add pullulan to such a composition in order to improve the effect thereof. Furthermore, and as is demonstrated by the inventors in Example 3, the stabilization of the hyaluronic acid by the alginic acid does not improve its efficacy as regards the insensible water losses.

Alginic acid and derivatives thereof (conjugated base, salts and esters) are natural polysaccharides obtained from a family of brown algae: Laminaria or Fucus. The alginate is a polymer formed of two monomers bonded together: mannuronate or mannuronic acid, some of which are acetylated and guluronate or guluronic acid. The bonding takes place via β-1,4. Alginates are used as thickeners, gelling agents, emulsifiers and stabilizers of the most diverse industrial products. They are also known as having water-retaining properties.

However, it has never been suggested to combine it with pullulan in order to obtain a product having high hydratability.

However, surprisingly, the inventors discovered that there was a synergy regarding the reduction of the insensible water losses between pullulan and a polysaccharide chosen from hyaluronic acid and alginic acid, optionally in the form of their respective salts or derivatives. The inventors discovered that this synergy was further strengthened when the three compounds were present. This combination, after application to the skin and/or mucous membranes, makes it possible to reduce the insensible water losses thereof, and thus to maintain the water content of the skin and/or mucous membranes. It was also discovered, very surprisingly, that the combination of these compounds, in particular of these 3 compounds, also increases the water content of the skin and/or mucous membranes. Thus, the combination of these compounds, in particular of these 3 compounds, has the advantage of providing a complete moisturizing effect, by the increase of the water content on the one hand and by maintaining the latter while reducing the water losses on the other hand. Furthermore, the inventors discovered, very surprisingly, that this combination, in particular when the 3 compounds are present and especially when they are added concomitantly, in particular in the form of a premix in a composition, also made it possible to increase and/or of prolong the content of cosmetic and/or pharmaceutical, in particular dermatological, active ingredients in the skin and/or the mucous membranes, in particular in the cutaneous epidermis.

The present invention therefore relates to the combination of pullulan or derivatives with a polysaccharide chosen from hyaluronic acid, a salt or derivative thereof, alginic acid, a salt or derivative thereof and a mixture of these polysaccharides. Advantageously, the polysaccharide consists of a mixture of hyaluronic acid or a salt or derivative thereof and of alginic acid or a salt or derivative thereof.

For the purposes of the present invention, the term “combination” is understood to mean the fact that the compounds are used together, preferably in the form of a premix. Advantageously, there is no covalent bond between the various compounds of the combination. For the purposes of the present invention, the expression “insensible water losses” is understood to mean the passive diffusion and insensible perspiration measured on a cutaneous or mucosal level. Also known as transepidermal water losses or TEWL, the insensible water losses may be measured by various methods, especially in vivo, in particular via an evaporimeter, a tewameter, especially in vitro, in particular by the open cylinder technique described in Example 1 and/or ex vivo. Thus, for example, the insensible water losses are measured by the technique described in Example 1 in an open cylinder and the reduction is determined with respect to an untreated control. This technique follows the example of those described in the publications by Lieb et al., “A new in vitro method for transepidermal water loss: a possible method for moisturizer evaluation”, J. Soc, Cosmet. Chem., March 1988, 39, 107-109 and Strussmann et al., “Water vapour permeability of skin care products in relation to molecular and environmental influence”, Int. Journal of Cosmetic Science, 1993, 15, 227-233. Nevertheless carried out in an oven and therefore in a dry atmosphere and at high temperature (45° C.), this test accentuates and amplifies the phenomenon of insensible water losses, enabling measurement under particularly harsh conditions.

For the purposes of the present invention, the expression “water content of the skin and/or mucous membranes” is understood to mean the amount of water contained in the epithelia, in particular the epidermis and/or the epithelium of the mucous membranes. This content thus expresses the state of hydration. There are various methods for measuring the water content of the skin and/or mucous membranes, especially in vivo and in particular via the corneometer, and/or by measuring the desquamation via the corneofix, or in vitro, especially via the measurement of the dielectric conductivity, in particular according to the method described in Example 4.

For the purposes of the present invention, the expression “content of cosmetic and/or pharmaceutical active ingredients in the skin and/or the mucous membranes”, is understood to mean the amount of cosmetic and/or pharmaceutical, in particular dermatological, active ingredients contained in the epithelia, in particular the epidermis and/or the epithelium of the mucous membranes. There are various methods for measuring the content of active ingredients in the skin and/or the mucous membranes. According to one preferred mode, this content will be measured ex vivo, in particular by studying the diffusion kinetics of the active ingredients in the skin and/or the mucous membranes. For example, it could be the study of penetration using a Franz cell, as described in Example 5.

For the purposes of the present invention, the expression “cosmetic and/or pharmaceutical ingredient(s)” is understood to mean one or more plant extracts and/or one or more natural or synthetic molecules and/or mixtures thereof intended for a cosmetic and/or pharmaceutical application. The cosmetic ingredients are especially defined by the international nomenclature of cosmetic ingredients (INCI).

The expression “cosmetic or pharmaceutical active ingredient” is understood to mean a cosmetic or pharmaceutical ingredient having a cosmetic and/or pharmaceutical efficacy. The pharmaceutical active ingredients correspond to pharmaceutical active principles. Categories and examples of cosmetic and/or pharmaceutical active ingredients are provided below.

Preferably, the cosmetic and/or pharmaceutical active ingredients according to the present invention are natural moisturizing factor compounds, such as amino acids, urea, hyaluronic acid and glycerol.

For the purposes of the present invention, the term “topical” is understood to mean the application of the combination and/or of the composition according to the invention to the surface of the skin and/or mucous membranes, especially by direct application or by vaporization.

According to the invention, the expression “mucous membranes” denotes especially the buccal, labial, nasal, ocular, anal and/or urogenital, in particular vaginal, mucous membranes.

For the purposes of the present invention, the expression “suitable cosmetic or pharmaceutical carrier” means that the composition or the components thereof are suitable for use in contact with human skin and/or mucous membranes with no undue toxicity, incompatibility, instability, allergic response, or their equivalents.

According to the invention, the “derivatives” are preferably the esterified derivatives and the organomineral silicon-based derivatives.

For the purposes of the present invention, the expression “esterified derivatives of pullulan, of hyaluronic acid or of alginic acid” is understood to mean all the derivatives obtained by single or multiple esterification of a primary or secondary alcohol function or of an acid function of pullulan, of hyaluronic acid or of alginic acid, and having, on the esterified part, a carbon-based chain comprising from 1 to 6 carbon atoms, advantageously a linear or branched alkyl chain.

For the purposes of the present invention, the expression “organornineral silicon-based derivatives of pullulan, of hyaluronic acid or of alginic acid” is understood to mean all the derivatives that contain at least one silanol (—SiOH) and are obtained by condensation of the pullulan, hyaluronic acid or alginic acid with a molecule from the family of silanes.

For the purposes of the present invention, the expression “salts of hyaluronic acid, or of alginic acid” is understood to mean the ionic compounds that result from the reaction for neutralization of the acid form of hyaluronic acid or of alginic, acid by an anion, especially inorganic ionic compounds, particular sodium, potassium, magnesium, chlorides, sulphates and phosphates.

Pullulan is produced from starch by the fungus Aureobasidium pullulans and may be obtained from various Aureobasidium pullulans ferments. According to the present invention, pullulan can be used in its aqueous, optionally saline form. The pullulan derivatives that can be used within the context of the present invention are the cosmetically and/or pharmaceutically acceptable, preferably dermatologically acceptable, derivatives, i.e. the derivatives that are non-toxic for administration to a human being, especially by topical application and that may be applied without risk and without causing an allergic or inflammatory reaction in particular on the skin. In one advantageous embodiment, the pullulan derivatives are chosen from the organomineral silicon-based derivatives customarily used in cosmetics such as, for example, those chosen from trimethylsiloxysilylcarbamoyl pullulan and trimethylsiiyl pullulan.

Advantageously in the combination, the pullulan is not in the derivative form. In particular, the pullulan has a molecular weight of less than 500 kDa, advantageously of around 200 kDa, Pullulan is commercially available (Chemaster International, China; Hayashibara, Japan).

The salts and derivatives of hyaluronic acid that can be used within the context of the present invention are the cosmetically and/or pharmaceutically acceptable, preferably dermatologically acceptable, salts or derivatives. Advantageously the salts of hyaluronic acid are chosen from hydrolysed calcium hyaluronate, hydrolysed sodium hyaluronate, potassium hyaluronate, sodium hyaluronate, sodium sulphated hyaluronate and mixtures thereof. Advantageously, it is sodium hyaluronate. In one advantageous embodiment, the hyaluronic acid derivatives are chosen from the derivatives customarily used in cosmetics such as, for example, those chosen from the esterified derivatives, in particular ascorbyl hyaluronate, benzyl hyaluronate, propylene glycol hyaluronate, sodium acetylated hyaluronate, sodium butyroyl hyaluronate, hydroxypropyltrimonium hyaluronate, the organomineral silicon derivatives, in particular dimethylsilanol hyaluronate and mixtures thereof. Advantageously in the combination, the hyaluronic acid is in the form of one of its salts. Advantageously, it is sodium hyaluronate. In particular the hyaluronic acid, the salts or esterified derivatives thereof have a molecular weight of greater than 20 kDa, advantageously between 50 and 800 kDa, advantageously between 250 and 450 kDa, Sodium hyaluronate is commercially available, especially from the companies Technidd Chemi-Tech, Wuhan Fortuna Chemical, Dalian Chem Imp. and Exp. Group, Afine Chemicals Limited, Javenech SA, Soliance, Maprecos, Landy Enterprise Limited, Chandigarh Medical Corporation. Kartik Enterprises, DSA Exports.

The salts and derivatives of alginic acid that can be used within the context of the present invention are the cosmetically and/or pharmaceutically acceptable, preferably dermatologically acceptable, salts or derivatives. Advantageously the salts of alginic acid are chosen from ammonium alginate, sodium alginate, calcium alginate, magnesium alginate, sodium sulphate alginate and potassium alginate. Advantageously, it is sodium alginate. In one advantageous embodiment, the alginic acid derivatives are chosen from the derivatives customarily used in cosmetics such as, for example, those chosen from the esterified derivatives, especially glyceryl alginate or propylene glycol alginate, the organomineral silicon derivatives of alginic acid, in particular siloxanetriol alginate or methylsilanol carboxymethyl theophylline alginate, and mixtures thereof. In particular the derivative is propylene glycol alginate. Advantageously in the combination, the alginic acid is in the form of one of its salts. Advantageously, it is sodium alginate. Advantageously, the alginic acid, salts or derivatives thereof, in particular esterified or organomineral silicon derivatives thereof, have a molecular weight between 10 and 600 kDa, preferably between 30 and 550 kDa, more preferably of 100 to 550 kDa. Sodium alginate is commercially available, especially from the companies Laserson S.A, Univar, Danisco Ingredients, BASE, BAM, Penta Manufacturing Company, Vevy Europe.

Advantageously, the alginic acid will be in the form of sodium alginate, propylene glycol alginate or mixtures thereof.

In one embodiment of the present invention, the weight ratio of pullulan, salts or esterified derivatives and polysaccharide is in the range 1/0.002-1/200, advantageously in the range 1/0.2-1/20, more advantageously still in the range 1/1-1/3.

In a first embodiment, the combination contains a mixture of pullulan, optionally in the form of derivative, and of hyaluronic acid, optionally in the form of salt or derivative, especially esterified derivative, advantageously in the form of sodium hyaluronate, Advantageously, for an amount x (in g) of pullulan, or derivatives, especially esterified derivatives, the amount of hyaluronic acid, salts or derivatives, especially esterified derivatives, will be between 0.001× and 1.00×. In particular, the weight ratio of pullulan, or derivatives, especially esterified derivatives/hyaluronic acid, salts or derivatives, especially esterified derivatives is in the range 1/0.001-1/100, advantageously in the range 1/0.1-1/10, more advantageously still it is 1/1. This embodiment has the advantage of long-term reducing the insensible water losses. A synergistic effect has especially been demonstrated in the experiment according to Example 1 carried out in vitro in an open cylinder on the reduction of the insensible water losses as from 5 hours after the application and this effect is very sizeable starting from 10 hours after the application of the combination and even more sizeable starting from 18 hours after application. This synergistic effect on the reduction of the insensible water losses is furthermore long-lasting.

In a second embodiment, the combination contains a mixture of pullulan, optionally in the form of derivative, and of alginic acid, optionally in the form of salt or derivative, especially esterified derivative, advantageously in the form of sodium alginate. Advantageously, for an amount x (in g) of pullulan, or derivatives, especially esterified derivatives, the amount of alginic acid, salts or derivatives, especially esterified derivatives, will be between 0.0011× and 100×. In particular, the weight ratio of pullulan, or derivatives, especially esterified derivatives/alginic acid, salts or derivatives, especially esterified derivatives is in the range 1/0.001-1/100, advantageously in the range 1/0.1-1/10, more advantageously still in the range 1/1-1/2. This embodiment has the advantage of long-term reducing the insensible water losses synergistically A synergistic effect has especially been demonstrated in the experiment according to Example 2 carried out in vitro in an open cylinder on the reduction of the insensible water losses as from 12 hours after the application and this effect is very sizeable starting from 15 hours after the application of the combination and even more sizeable starting from 18 hours after application. This synergistic effect on the reduction of the insensible water losses is furthermore long-lasting.

In a third embodiment, the combination contains a mixture of pullulan, optionally in the form of derivative, of hyaluronic acid, optionally in the form of salt or derivative, especially esterified derivative, advantageously in the form of sodium hyaluronate, and of alginic acid, optionally in the form of salt or derivative, especially esterified derivative, advantageously in the form of sodium alginate. This embodiment has the advantage of reducing the insensible water losses very effectively and rapidly. Thus, a synergistic effect has especially been demonstrated in the experiment according to Example 3 carried out in vitro in an open cylinder on the reduction of the insensible water losses from 4 hours after the application of the combination and this with an intensity almost identical to the hyaluronic acid but in a much more long-lasting manner than the latter.

According to this third embodiment, advantageously, the combination therefore comprises pullulan, sodium hyaluronate and sodium alginate. Advantageously, the weight ratio of pullulan, optionally in the form of derivatives/hyaluronic acid, salts or esterified derivatives/alginic acid, salts or esterified derivatives is in the range 1/0.001/0.001 to 1/100/100, more advantageously in the range 1/0.1/0.1 to 1/10/10, even more advantageously in the range 1/1/1 to 1/10/10, and more preferably in the range 1/1/1 to 1/2/5. According to a preferred mode, this ratio is around 1/1/2, more preferably it is 1/1/2.

Besides the properties of limiting the insensible water losses, the inventors have shown in Example 3 that this third embodiment makes it possible to increase the water content of the skin and/or mucous membranes. Thus, the inventors have demonstrated that the combination of the three compounds according to the third embodiment makes it possible both to increase the water content and to reduce the insensible water losses, Which is particularly advantageous for a moisturizing effect that is complete, immediate and long-lasting over time. The combination according to the third embodiment is very particularly suitable as a moisturizing agent for the skin and/or the mucous membranes.

Furthermore, the inventors have demonstrated that the combination according to the third embodiment had the property of increasing and/or prolonging the content of cosmetic and/or pharmaceutical active ingredients in the skin and/or the mucous membranes, in a long-lasting manner (Example 5). This advantageous property of said combination is explained by the formation of a multilayered molecular network, corresponding to a molecular lattice with formation of hydrogen bonds, when the 3 compounds pullulan, optionally in the form of derivatives, hyaluronic acid, salts or esterified derivatives, and alginic acid, salts or esterified derivatives are combined concomitantly, preferably in the form of a premix, preferably in a ratio that is in the range 1/1/1 to 1/10/10, and more preferably in the range 1/1/1 to 1/2/5, advantageously a ratio of 1/1/2 (Example 6).

The present invention also relates to the use of the combination according to the present invention as a moisturizing agent for the skin and/or mucous membranes, advantageously having an immediate and long-lasting action. Indeed, the inventors observed that the combination comprising two or three components had an intensified activity on the reduction of the water losses and therefore on maintaining the hydration, due to the synergy between its various components, for a long duration. Indeed, the synergistic effect appears even more sizeable as a function of time. Advantageously this activity last at least 5 hours, preferably 10 hours, more preferably 24 hours and may even range up to 48 hours. Moreover, the inventors observed that this combination, in particular when it comprises the three components, had an intensified activity for the increase of the water content, in a long-lasting manner, and this in particular due to the formation of a molecular network when the 3 compounds pullulan, optionally in the form of derivatives, hyaluronic acid, salts or esterified derivatives, and alginic acid, salts or esterified derivatives are combined concomitantly, preferably in the form of a premix, preferably in a ratio that is in the range 1/1/1 to 1/10/10, and more preferably in the range 1/1/1 to 1/2/5, advantageously a ratio of 1/1/2 (Example 6). Advantageously this activity lasts at least 3 hours, more preferably 10 hours. The present invention therefore relates to the use of the combination according to the present invention for reducing the insensible water losses and/or for increasing the water content of the skin and/or mucous membranes.

The present invention additionally relates to the use of the combination according to the present invention for increasing and/or prolonging the content of cosmetic and/or pharmaceutical active ingredients in the skin and/or the mucous membranes and/or maintaining it over time.

The combination according to the invention, and preferably in the form of the 3 compounds is preferably used alone or in the form of a mixture of cosmetic or pharmaceutical ingredients contained in a cosmetic or pharmaceutical, especially dermatological, carrier suitable for its formulation and/or its integration into a cosmetic or pharmaceutical, especially dermatological, composition. The combination may also be used in a cosmetic or pharmaceutical, especially dermatological, composition, i.e. combined with a suitable cosmetic or pharmaceutical carrier, especially suitable dermatological carrier, and preferably in topical application. Preferably, said combination consists of a mixture of the 3 compounds pullulan, optionally in the form of derivatives, hyaluronic acid, salts or esterified derivatives, and alginic acid, salts or esterified derivatives, preferably in a ratio that is in the range 1/1/1 to 1/10/10, and more preferably in the range 1/1/1 to 1/2/5, advantageously a ratio of 1/1/2 (Example 6).

The combination according to the invention may also be used in the form of a cleansing composition, in particular a detergent composition, such as for example a multi-purpose cleanser, a soap and/or a washing-up liquid.

According to a first embodiment, the present invention relates to a mixture of cosmetic or pharmaceutical, in particular dermatological, ingredients advantageously intended to be incorporated into a cosmetic or pharmaceutical, in particular dermatological, composition comprising the combination according to the present invention, preferably in the form of the 3 compounds and a suitable cosmetic or pharmaceutical carrier. Advantageously, the carrier for the mixture of cosmetic or pharmaceutical ingredients is and/or contains water.

Advantageously the combination is then included in the mixture of cosmetic or pharmaceutical, in particular dermatological, ingredients in a content of 0.001% to 20% by weight of dry matters relative to the total weight of the mixture of cosmetic or pharmaceutical ingredients, more advantageously between 0.01% and 10% by weight, even more advantageously between 0.1% and 5% by weight, in particular between 0.25% and 3% by weight, more particularly between 1% and 3%, even more particularly 1% by weight. According to one advantageous mode, the mixture of cosmetic or pharmaceutical, in particular dermatological, ingredients contains:
pullulan, a cosmetically or pharmaceutically acceptable derivative thereof originating from the combination according to the present invention in a content between 0.0001% and 10% by weight of dry matters relative to the total weight of the mixture of ingredients, more advantageously between 0.001% and 5% by weight, even more advantageously between 0.01% and 3% by weight, in particular between 0.1% and 1% by weight, even more particularly between 0.25% and 0.5% by weight, very particularly 0.25% by weight; and
hyaluronic acid, a cosmetically or pharmaceutically acceptable salt or derivative thereof originating from the combination according to the present invention in a content between 0.0001% and 10% by weight of dry matters relative to the total weight of the mixture of ingredients, advantageously between 0.001% and 5% by weight, more advantageously between 0.01% and 3% by weight, in particular between 0.1% and 1% by weight, more particularly between 0.1% and 1% by weight, even more particularly between 0.25% and 0.5% by weight, very particularly 0.25% by weight; and/or
alginic acid, a cosmetically or pharmaceutically acceptable salt or derivative thereof originating from the combination according to the present invention in a content between 0.0001% and 10% by weight of dry matters relative to the total weight of the mixture of ingredients, advantageously between 0.001% and 5% by weight, more advantageously between 0.01% and 3% by weight, even more advantageously between 0.1% and 2% by weight, even more particularly 0.5% by weight.

According to one preferred mode, the mixture of cosmetic or pharmaceutical ingredients contains the combination of pullulan or a derivative thereof, of hyaluronic acid, or a salt or derivative thereof and of alginic acid or of a salt or derivative thereof, preferably in a ratio between 1/1/1 and 1/10/10, preferably between 1/1/1 and 1/2/5, more preferably in a ratio of 1/1/2.

The mixture of cosmetic or pharmaceutical, in particular dermatological, ingredients may additionally comprise another moisturizing agent, advantageously having a synergistic or complementary effect. It is in particular chosen from trehalose, serine, urea and mixtures thereof.

According to one particularly advantageous mode, the mixture of cosmetic or pharmaceutical, in particular dermatological, ingredients comprises at least the combination according to the invention, preferably of the 3 compounds, and also serine, trehalose, urea and water.

The mixture of cosmetic or pharmaceutical, in particular dermatological, ingredients may be used in a cosmetic or pharmaceutical, in particular dermatological, composition preferably in a content by weight of dry matters relative to the total weight of the composition between 0.1% and 10%, advantageously between 0.1% and 5%, in particular between 1% and 3%.

In a second embodiment of the present invention, the present invention relates to a cosmetic or pharmaceutical, in particular dermatological, composition intended for topical administration comprising the combination or the mixture of cosmetic or pharmaceutical ingredients according to the present invention.

Thus in this second embodiment of the present invention, the combination is included in a cosmetic or pharmaceutical, in particular dermatological, composition in particular intended to be administered to a human being, preferably by topical, preferably cutaneous, application. Advantageously the combination according to the present invention is then included in the composition in a content between 0.0001% and 20% by weight of dry matters relative to the total weight of the composition, more advantageously between 0.005% and 10% by weight, even more advantageously between 0.01% and 5% by weight, in particular between 0.02 and 1% by weight, more particularly 0.03% by weight. The cosmetic or pharmaceutical, in particular dermatological, composition preferably also contains a cosmetic and/or pharmaceutical, in particular dermatological, carrier and/or excipient.

According to one advantageous mode, the cosmetic or pharmaceutical, in particular dermatological, composition contains:
pullulan, a cosmetically or pharmaceutically acceptable derivative thereof in an amount between 0.0001% and 10% by weight of dry matters relative to the total weight of the composition, and advantageously an amount between 0.001% and 5% by weight, more advantageously between 0.001% and 3% by weight, in particular between 0.005% and 0.1% by weight, more preferably between 0.007% and 0.02% by weight; and
hyaluronic acid, a cosmetically or pharmaceutically acceptable salt or derivative thereof in a content between 0.0001% and 10% by weight of dry matters relative to the total weight of dry matters of the composition, advantageously between 0.001% and 5% by weight, more advantageously between 0.001% and 3% by weight, in particular between 0.005% and 0.1% by weight, more preferably between 0.007% and 0.02% by weight; and
alginic acid, a cosmetically or pharmaceutically acceptable salt or derivative thereof in a content between 0.0001% and 10% by weight of dry matters relative to the total weight of the composition, advantageously between 0.001% and 5% by weight, more advantageously between 0.001% and 1% by weight, in particular between 0.007% and 0.2% by weight, even more particularly between 0.01% and 0.1% by weight.

According to one preferred mode, the cosmetic or pharmaceutical composition contains the combination of pullulan or a derivative thereof, of hyaluronic acid, or a salt or derivative thereof and of alginic acid or a salt or derivative thereof, preferably in a ratio of 1/1/2.

The composition according to the present invention, in the form of cosmetic or pharmaceutical composition or of a mixture of cosmetic or pharmaceutical ingredients, may be in any galenic form conventionally used for a topical application, such as liquid or solid forms or even in the form of a pressurized liquid. They may especially be formulated in the form of an aqueous or oily solution, a cream or an aqueous gel or an oily gel, especially in a pot or in a tube, especially a shower gel, a shampoo, a milk, an emulsion, a hydrogel, a microemulsion or a nanoemulsion, especially of oil-in-water or water-in-oil or multiple or silicone type, a serum, a lotion, especially in a glass or plastic bottle or in a measuring bottle or in an aerosol, a vial, a liquid soap, a paste, a dermatological bar, an ointment, a foam, a mask, a patch, an anhydrous product, preferably that is liquid, pasty or solid, for example in the form of a rod, especially in stick or powder form, especially for makeup. In particular the composition is in the form of a serum, a lotion, a cream, a milk, an ointment, a paste, a foam, an emulsion, a hydrogel, a shower gel, a mask, a stick, a patch, or makeup powders, advantageously a cream or a lotion.

The compositions according to the invention may contain any suitable solvent and/or any suitable carrier and/or any suitable excipient, optionally combined with other compounds of interest. They may especially contain a cosmetically or dermatologically acceptable excipient chosen from surfactants, preserving agents, buffers, swelling agents, chelating agents, biocidal agents, denaturants, opacifiers, pH adjusters, reducing agents, stabilizers, emulsifiers, thickeners, gelling agents, film forming polymers, solvents, fillers, bactericides, odour absorbers, matifying agents, conditioners, texturing agents, shine agents, pigments, dyes, fragrances and chemical or mineral sunscreens, trace elements, essential oils. These combinations are also covered by the present invention. The CTFA Cosmetic Ingredient Handbook, Second Edition (1992) describes various cosmetic and pharmaceutical ingredients commonly used in the cosmetic and pharmaceutical industry, which are in particular suitable for topical use.

Various cosmetic and/or pharmaceutical, in particular dermatological, active ingredients, alone or as a mixture, can be delivered topically using the combination according to the present invention.

The compositions according to the invention may thus contain cosmetic or pharmaceutical, especially dermatological, active ingredients resulting in a complementary or optionally synergistic effect such as moisturizing active agents, anti-ageing active agents, free-radical scavenging active agents and/or thermal waters. They may thus be for example skin depigmenting, lightening or anti-pigmenting agents, skin-coloring or pro-pigmenting agents, NO-synthase inhibitors, anti-seborrheic agents for the care of oily skin, agents for stimulating the synthesis of dermal or epidermal macromolecules, especially of the extracellular matrix, and/or for preventing their degradation, agents tear stimulating fibroblast or keratinocyte proliferation and/or keratinocyte differentiation, muscle relaxants or dermo-decontracting agents, antimicrobial agents, tensioning agents, anti-pollution agents or free-radical scavengers, soothing, calming or relaxing agents, agents that act on the microcirculation to improve the radiance of the complexion, in particular of the face, photoprotective agents, desquamating agents, wound-healing agents, slimming agents, anti-ageing agents or optionally other moisturizing agents and/or agents that strengthen the epidermal barrier in order to further intensify the moisturizing activity.

The moisturizing, emollient or humectant active agents resulting in a complementary or optionally synergistic effect are those which strengthen the barrier function and reduce the insensible water losses and/or those which increase the water content of the skin and/or mucous membranes or stimulate the secretory activity of the sebaceous glands and/or stimulate the synthesis of aquaporin to improve the circulation of water in the cells. By way of nonlimiting example, mention may be made of the following active agents: serine, urea and derivatives thereof, the products sold under the name Marine Filling Spheres™, Advances Moisturizing Complex™. Hyaluronic Filling Spheres™, Vegetal Filling Spheres™, Osmogelline™, Micropatch™, alkyl celluloses, lecithins, sphingoid-based compounds, ceramides, phospholipids, cholesterol and derivatives thereof, glycosphingolipids, phytosterols (stigmasterol and beta-sitosterol, campesterol), essential fatty acids, 1,2-diacylglycerol, 4-chromanone, pentacyclic triterpenes such as ursolic acid, petroleum jelly, lanolin, sugars in particular trehalose and derivatives thereof, rhamnose, fructose, maltose, lactose, erythritol, mannitol, D-xylose and glucose, adenosine and derivatives thereof, sorbitol, polyhydric alcohols, advantageously C2-C6, and more advantageously C3-C6 polyhydric alcohols, such as glycerin, propylene glycol, 1,3-butylene glycol, dipropylene glycol, diglycerin, polyglycerin and mixture thereof, glycerol and derivatives thereof, glyceryl polyacrylate, sodium lactate, pentanediol, serine, lactic acids, AHA, BHA, sodium pidolate, xylitol, sodium lactate, ectoine and derivatives thereof, chitosan and derivatives thereof, collagen, plankton, steroid derivatives (including DHEA, its 7-oxidized and/or 17-alkylated derivatives and sapogenins), methyl dihydrojasmonate, vitamin D and derivatives thereof, an extract of Malva sylvestres or an extract of Centelia asiatica, acrylic acid homopolymers, beta-glucan and in particular sodium carboxymethyl beta-glucan, a C-glycoside derivative such as those described in application WO 02/051828, a musk rose oil, an extract of the microalga Prophyridium cruentum enriched with zinc sold by Vincience under the name Algualane Zinc™, arginine, acetyl hexapeptide sold by Lipotech under the name Diffuporine™, Viola tricolor hydrolysate sold by Silab under the name Aquaphyline™.

The other active agents resulting in a complementary effect may nonlimitingly be chosen from the list of skin depigmenting, lightening or anti-pigmenting agents, skin-coloring or pro-pigmenting agents, NO-synthase inhibitors, anti-seborrheic agents for the care of oily skin, agents for stimulating the synthesis of dermal or epidermal molecules and macromolecules and/or for preventing their degradation, agents for stimulating fibroblast or keratinocyte proliferation and/or keratinocyte differentiation, muscle relaxants or dermo-decontracting agents, antimicrobial agents, tensioning agents, anti-pollution agents or free-radical scavengers, soothing, calming or relaxing agents, agents that act on the microcirculation to improve the radiance of the complexion, in particular of the face, agents for stimulating the synthesis of the extracellular matrix, photoprotective agents, desquamating agents, wound-healing agents and/or anti-ageing agents.

Examples of these agents that can be used in the compositions according to the present invention and/or that can be delivered by the combination according to the present invention are the following:

As examples of wound-healing agents, mention may especially be made of: allantoin, urea, certain amino acids, for instance hydroxyproline, arginine, serine, yeast extracts, chitosan and derivatives, for instance chitosan glutamate, common yarrow extracts, folic acid, beta-glucan and derivatives, shea butter and its purified fractions, modified exopolysaccharides and alkylsulphonated polyaminosaccharides.

The active agent may also be chosen from anti-ageing agents, that is to say agents especially having a cutaneous barrier restructuring effect, agents for preventing and/or reducing glycation of the skin proteins, in particular resveratrol, in particular dermal proteins, such as collagen, active agents for stimulating the energy metabolism of cells and mixtures thereof, an agent having an overall anti-ageing action, especially an anti-pigmentation marks action, in particular niacinamide or vitamin B3 and derivatives. The agent having a cutaneous barrier restructuring effect may be chosen from a yeast extract for instance Relipidium™ from BASF Beauty Care Solutions France SAS, sphingosines, for instance salicyloyl sphingosine, a mixture of xylitol, xylityl polyglycoside and xylitan, extracts of Solanacea plants, for instance Lipidessence™ from BASF Beauty Care Solutions France SAS and mixtures thereof. Mention may also be made especially of ceramides, sphingoid-based compounds, glycosphingolipids, phospholipids, cholesterol and derivatives thereof, phytosterols, essential fatty acids, diacylglycerol, 4-chromanone and chromone derivatives and mixtures thereof, vitamin B5 or pantothenate and derivatives.

The active agent for stimulating the energy metabolism of cells may be chosen, for example, from biotin, a mixture of sodium, manganese, zinc and magnesium salts of pyrrolidone carboxylic acid, a mixture of zinc, copper and magnesium gluconate, and mixtures thereof.

By way of example of skin depigmenting, lightening or anti-pigmenting agents that can be used within the context of the present invention, mention may especially be made of the following compounds: kojic acid, ellagic acid, ferulic acid and derivatives thereof, arbutin, hydroquinone, calcium pantothenosulphonate, boldine, diacetyl boldine, ascorbic acid and derivatives thereof, especially ascorbyl glucoside.

The skin-coloring or pro-pigmenting agents may especially be chosen from agents for promoting melanogenesis, artificial skin-coloring agents, and mixtures thereof.

The agents for promoting melanogenesis are especially chosen from:
melanin biosynthesis substrates, especially L-tyrosine and derivatives thereof, or L-dihydrophenylalanine;
agents capable of stimulating melanin synthesis, and in particular the complexes of metal ions such as copper and of peptones such as a protein hydrolysate originating from soybean, collagen or casein, as described in patent U.S. Pat. No. 5,698,184;
the agents capable of stimulating the activity or expression of tyrosinase, optionally by increasing the level of intracellular cAMP such as, especially pro-opiomelanocortic peptides; α-MSHs or α-MSH analogues; or MC1R receptor agonists (U.S. Pat. No. 5,683,981 or WO 98/25584), cAMP analogues, forskolin and xanthine bases (for example caffeine or theophylline) or by activation of protein kinase C, such as diacylglycerols or else psoralens,
the agents capable of stimulating the transfer of the melanosomes from the melanocytes to the keratinocytes, for example by stimulating the PAR-2 receptors such as catechin polyphenols, in particular, catechin, epicatechin, gallocatechin and epigallocatechin, the salts and esters thereof, in monomeric or oligomeric form, and also plant extracts containing them, in particular extracts of green tea.

The artificial skin-coloring agents are especially chosen from:
self-tanning agents, such as isatin, alloxan, ninhydrin, glyceraldehyde, the mesotartaric aldehyde, glutaraldehyde, erythrulose, dihydroxyacetone (DHA),
additional coloring agents such as sorghum extracts and an extract of Cichorium intybus, in particular combined with an extract of Gymnema sylvestre or Muira Puama, sold under the name QUICKSUN by BASF Beauty Care Solutions France SAS.

Examples of NO-synthase inhibitors are especially an extract of a plant of the species Vitis vinifera.

The anti-seborrheic agent in the composition according to the invention may be a 5α-reductase reductase inhibitor, such as retinoids, sarcosine, zinc salts, in particular zinc gluconate, zinc salicylate, azelaic acid and/or derivatives thereof, and/or mixtures thereof and an extract of Orthosiphon stamineus sold under the name MAT XS™ Bright by BASF Beauty Care Solutions France SAS.

The composition may also contain a sebum-absorbing agent, in particular a talc and/or an absorbent polymer, an antibacterial agent especially those described in patent application FR 2863893, and in particular an extract of Boldo, such an extract especially being sold by the Applicant under the name Betapur™, a comedolytic agent in particular the retinoic acid and a derivative thereof such as isotretinoin, adapalene and/or 13-cis-retinoic acid and benzoyl peroxide, a local antibiotic agent, in particular erythromycin and/or clindamycin phosphate and mixtures thereof.

Among the active agents for stimulating the synthesis of dermal macromolecules, or for preventing their degradation, mention may be made of those that act as:
an agent for stimulating the synthesis of fibronectin, in particular a maize extract, such an extract being especially sold by the Applicant under the name Deliner™ and palmitoyl pentapeptide sold by the company SEDERMA under the trade name Matrixil®,
an agent for protecting the fibroblast growth factor (FGF2) of the extracellular matrix against the degradation thereof and/or the denaturing thereof, especially an extract of Hibiscus abelmoschus as described in the patent application in the name of the Applicant filed under the number FR 0654316 and/or an agent for stimulating fibroblast growth, for example a fermented soybean extract containing peptides, known under the name Phytokine™ sold by the Applicant and also described in patent application EP 1 119 344 B1 (Laboratoires Expanscience), and preferably a combination of these two extracts;
an agent for stimulating the synthesis of laminin, in particular an extract of malt modified by biotechnology, such an extract being especially sold by the Applicant under the name Basaline™;
an agent for stimulating the expression and/or the activity of hyaluronan synthase-2 (HAS2) such as the plant extracts described in patent application FR 2 893 252 A1 and in particular an aqueous extract of galangal (Alpinia galangal);
an agent for stimulating the synthesis of lysyl oxidase-like (LOXL) such as an extract of Geophila cordifolia and those described in patent application FR 2855968, and in particular an extract of dill;
an agent for stimulating the synthesis of intracellular ATP, especially an extract of the alga Laminaria digitata;
an active agent for stimulating the synthesis of glycosaminoglycans, such as the fermentation product of milk;
a collagen-stimulating active agent such as retinol and/or vitamin C;
an active agent for inhibiting metalloproteinases (MMPs) such as, more particularly MMP 1, 2, 3 or 9, for instance retinoids and derivatives, oligopeptides and lipopeptides, lipoamino acids, the malt extract sold by BASF Beauty Care Solutions France under the trade name Collalift®, the hydrolysed potato extract sold under the name Extracellium™ by BASF Beauty Care solutions France SAS; lycopene; isoflavones, quercetin, kaempferol, apigenin.

The agents for stimulating keratinocyte proliferation that can be used in the composition according to the invention especially comprise retinoids such as retinol and its esters, including retinyl palmitate, and phloroglucinol. The agents for stimulating keratinocyte differentiation comprise, for example, minerals such as calcium and lignans such as secoisolariciresinol and also the extract of Achillea millefolium sold under the name Neurobiox™ by BASF Beauty Care Solutions France.

The muscle relaxants or dermo-decontracting agents that can be used in the composition according to the invention comprise manganese gluconate, Diazepam, certain secondary and tertiary carbonyl amines, adenosine, and also sapogenins.

The antimicrobial agents that can be used in the composition according to the invention may especially be chosen from the 2,4,4′-trichloro-T-hydroxydiphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, undecylenic acid and its salts, benzoyl peroxide, 3-hydroxybenzoic acid, 4-hydroxybenzoic acid, phytic acid. N-acetyl-L-cysteine acid, lipoic acid, azelaic acid and its salts, arachidonic acid, resorcinol, octoxyglycerin, octanoylglycine, caprylyl glycol, 10-hydroxy-2-decanoic acid, farnesol and phytosphingosines, and mixtures thereof.

Among the tensioning agents that can be used in the composition according to the present invention, mention may especially be made of synthetic polymers, such as polyurethane latex or acrylic latex; polymers of natural origin, especially polysaccharides in the form of starch or in the form of carrageenans, alginates, agars, gellans, cellulose polymers and pectins; plant proteins and protein hydrolysates from soybean; mixed silicates; wax microparticles; colloidal particles of inorganic filler chosen, for example, from: silica, silica-alumina composites; and also mixtures thereof.

The composition may comprise agents referred to as anti-pollution agents, in particular ozone-trapping agents which are for example vitamin C and its derivatives including ascorbyl glucoside; phenols and polyphenols, in particular tannins, ellagic acid and tannic acid; epigallocatechin and natural extracts containing it, in particular extracts of green tea; anthocyans; phenol acids, the stilbenes, resveratrol; active agents for trapping monocyclic or polycyclic aromatic compounds, tannins such as ellagic acid and indole derivatives and/or active agents for trapping heavy metals such as EDTA, free-radical scavengers such as vitamin E and its derivatives such as tocopheryl acetate; bioflavonoids; coenzyme Q10 or ubiquinone.

As soothing agents that can be used in the composition according to the invention, mention may be made of: pentacyclic triterpenes, ursolic acid and its salts, oleanolic acid and its salts, betulinic acid and its salts, the salts of salicylic acid and in particular zinc salicylate, bisabolol, allantoin, omega-3 unsaturated oils, cortisone, hydrocortisone, indomethacin and betamethasone, anti-inflammatory active agents, and especially those described in application FR 2847267, in particular the Pueraria lobata root extract sold under the name Inhipase® by BASF Beauty Care Solutions France SAS, and extracts of Theobroma cacao.

The active ingredients that act on the microcirculation (vasoprotectives or vasodilators) may be chosen from flavonoids, ruscogenins, nicotinates, and essential oils.

The photoprotective active ingredients or UVA and/or UVB screening agents that can be used according to the present invention are especially the UV-A-active and/or UV-B-active photoprotective agents such as para-aminobenzoic acid derivatives especially UVINUL P25 by BASF, salicylic derivatives, in particular homosalate alone or in combination with titanium oxides,

dibenzoylmethane derivatives, cinnamic derivatives, diphenyl acrylate derivatives, including octocrylene sold especially under the trade name UVINUL N539 by BASF, benzophenone derivatives, especially benzophenone-1 sold especially under the trade name UVINUL 400 by BASF, benzylidene camphor derivatives, benzimidazole derivatives, triazine derivatives, including ethylhexyl triazone sold especially under the trade name UVINUL T150 by BASF, benzotriazole derivatives, anthranilic derivatives, imidazoline derivatives, benzalmalonate derivatives, 4,4-diarylbutadiene derivatives, and mixtures thereof.

The expression “desquamating agent” is understood to mean any compound capable of acting:
either directly on desquamation by promoting exfoliation, such as β-hydroxy acids, in particular salicylic acid and its derivatives, in particular ester derivatives (including 5-n-octanoylsalicylic acid), fruit acids, α-hydroxy acids, such as salicylic acid, glycolic acid, citric acid, lactic acid, tartaric acid, malic acid or mandelic acid, gentisic acid or its esters in particular tocopheryl gentisate, oligofucoses, cinnamic acid, resveratrol and certain jasmonic acid derivatives, and/or derivatives thereof and/or mixtures thereof,
or on the enzymes involved in desquamation or the degradation of corneodesmosomes, glycosidases, agents for chelating mineral salts: EDTA, the aminosulphonic compounds, sodium methylglycinediacetate sold by BASF under the trade name TRILON™, honey, sugar derivatives such as O-octanoyl-6-D-maltose and N-acetylglucosamine.

The active agents that provide a well-being effect such as those mimicking the effects of beta-endorphins in order to improve the barrier function of the skin, such as those cited in patent application US 2006/069032; the active agents that stimulate the synthesis of beta-endorphins such as an extract of the plant Tephrosia purpurea.

The slimming active agents may especially be chosen from: the agents that inhibit the lipoprotein lipase such as those described in patent US 2003/086949 (Coletica) and in particular an extract of Peruvian liana (Uncaria tomentosa); draining active agents, especially hesperitin laurate (Flavagrum®), or quercitin caprylate (Flavenger®); the agents that inhibit the phosphodiestarase enzyme, agents that activate adenylate cyclase, cAMP and/or the active agents capable of trapping spermine and/or spermidine. Mention may be made, by way of example of these active agents of a Coleus forskohlii root extract, an extract of Cecropia obtusa or of Uva lactuca, caffeine, forskolin, theophylline, theobromine and/or their derivatives, a hydrolysed kappa-carrageenan product known as Slimexcess™ sold by BASF Beauty Care Solutions France SAS and/or mixtures thereof.

All these cosmetic or pharmaceutical active ingredients may be used with the combination according to the present invention, in particular for increasing their content in the skin and/or mucous membranes and prolonging the effects thereof, especially via a retarding effect on their percutaneous diffusions by way of the molecular network formed when the 3 compounds according to the invention are combined concomitantly, and preferably in the form of a premix, in the compositions according to the invention.

Preferably, the compositions according to the invention in the form of a mixture of cosmetic or pharmaceutical ingredients or of cosmetic or pharmaceutical composition contain at least cosmetic or pharmaceutical, in particular dermatological, active ingredients, and preferably from one to five cosmetic ingredients, more preferably from one to three cosmetic or pharmaceutical, in particular dermatological, active ingredients. Preferably, the active ingredient is chosen from those mentioned previously, advantageously from anti-ageing and moisturizing agents, and more preferably from urea, trehalose, serine, taurine and/or a derivative thereof, inositol, betaine, in particular at least two of these compounds, preferably chosen from serine, urea and trehalose and advantageously three of these compounds, preferably serine, urea and trehalose.

The present invention also relates to the use of the cosmetic composition according to the present invention for reducing the insensible water losses, thr increasing the water content of the skin and/or mucous membranes, for maintaining or intensifying the state of hydration of the skin and/or mucous membranes, and/or for preventing or slowing down the appearance of the signs of cutaneous and/or mucosal dryness, in particular over at least 10 hours, advantageously 24 hours, in particular up to 48 hours and/or for treating cutaneous and/or mucosal dryness conditions such as the squamous states and/or itching and/or tautness associated with dry skin or mucous membranes and/or for preventing or reducing the appearance of wrinkles linked to cutaneous dryness, and/or for improving the comfort of dry skin and/or mucous membranes, and/or for treating skin and/or mucous membranes having an appearance that is rough to look at and/or to the touch.

The combination according to the invention, especially in the form of a cosmetic or pharmaceutical, in particular dermatological, composition, is therefore very particularly suitable for caring for and/or treating dry skin and/or mucous membranes, aggressed, sensitive, sensitized, impaired, intolerant, senile, fragile or reactive skin and/or mucous membranes. The combination is also very particularly suitable for use on buccal, labial, ocular, genital, especially vaginal, mucous membranes, alone or in the form of a cosmetic or dermatological composition, in particular for improving the state of hydration thereof. The combination alone or in the form of a cosmetic or pharmaceutical composition or a mixture of cosmetic or pharmaceutical ingredients is also very particularly suitable for producing ophthalmic care compositions.

The combination alone or in the form of a cosmetic or pharmaceutical composition or a mixture of cosmetic or pharmaceutical ingredients may be applied especially to the face, neck, bust, body, hands, feet, scalp, eyes and/or lips.

The present invention also relates to a pharmaceutical, in particular dermatological, composition according to the present invention for use in the treatment and/or the prevention of fissures and/or scurf or pityriasis alba and/or cracks and/or atopic dermatitis and/or ichthyosis and/or conditions of dryness of the skin and/or mucous membrane that accompany cutaneous and/or mucosal pathologies such as eczema.

Lastly, the present invention relates to a cosmetic care method characterized in that it comprises the application to at least one concerned region of the skin and/or mucous membranes of the face or body, in particular aggressed, sensitive, sensitized, impaired, intolerant, senile, fragile or reactive skin and/or mucous membranes, of the combination according to the present invention or of a cosmetic composition or of a mixture of cosmetic or pharmaceutical ingredients comprising, as active agent, the combination as defined previously, for maintaining or intensifying the state of hydration of the skin, in particular aggressed, sensitive, sensitized, impaired, intolerant, senile, fragile or reactive skin and/or mucous membranes, and/or for preventing or slowing down the appearance of the signs of cutaneous and/or mucosal dryness, in particular over at least 10 hours, advantageously 24 hours, more advantageously up to 48 hours and/or for treating cutaneous and/or mucosal dryness conditions such as the squamous states and/or itching and/or tautness associated with dry skin and/or mucous membranes and/or for preventing or reducing the appearance of wrinkles linked to cutaneous dryness, and/or for improving the comfort of dry skin and/or mucous membranes, and/or for treating skin and/or mucous membranes having an appearance that is rough to look at and/or to the touch.

The invention also relates to a method for treating and/or preventing and/or reducing the occurrence of fissures and/or scurf or pityriasis alba and/or cracks and/or atopic dermatitis and/or ichthyosis and/or conditions of dryness of the skin and/or mucous membrane that accompany cutaneous and/or mucosal pathologies such as eczema comprising the application, to at least one concerned region of the skin and/or mucous membranes of the face or body of a patient having need thereof, of the combination according to the present invention or of a pharmaceutical composition or of a mixture of pharmaceutical ingredients comprising, as active agent, the combination as defined previously.

Thus, the combination according to the present invention being able to be used with cosmetic and/or pharmaceutical, in particular dermatological, active ingredients to increase the content and the duration of action thereof in the epidermis and/or mucous membranes.

Thus, the composition according to the present invention containing this combination and optionally these active ingredients will be able to be used in the following applications, which are a function of the possible uses of these active agents: antioxidant, anti-inflammatory, anti-wrinkle/anti-ageing, lifting/tensioning/smoothing care product, for the radiance of the complexion, promoting cell replication, that regulate seborrhea, that are matifying, that regulate the size of the pores of the skin, that are wound-healing, moisturizing, soothing, slimming, anti-UV/sunscreen, aftersun/regenerating, makeup products.

Lastly, the present invention relates to a cleansing composition, in particular detergent composition, comprising the combination according to present invention.

The invention will be better understood on reading the description of the figures and examples which follow.

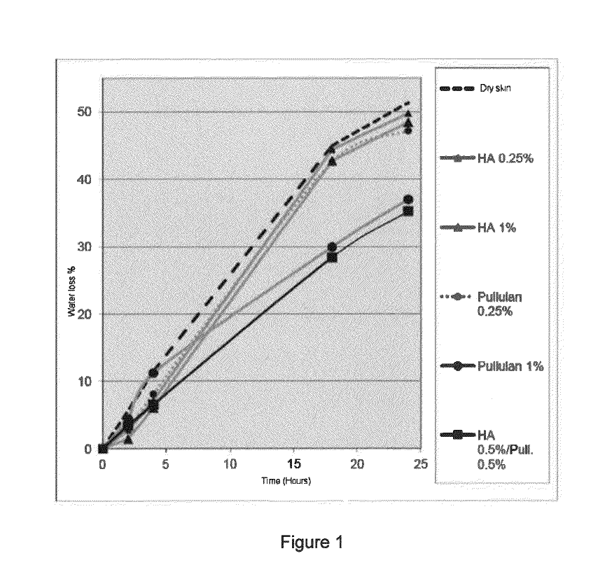

FIG. 1 represents the comparison between the measurement of the insensible losses as a function of time obtained with a composition containing the combination according to the invention (0.5% by weight of pullulan and 0.5% by weight of hyaluronic acid in the form of sodium hyaluronate: HA 0.5%/Pull. 0.5%) and the measurement of the insensible losses as a function of time obtained with:

a composition containing 0.25% by weight of pullulan (Pullulan 0.25%),

a composition containing 1% by weight of pullulan (Pullulan 1%),

a composition containing 0.25% by weight of hyaluronic acid, in the form of sodium hyaluronate (HA 0.25%), and

a composition containing 1% by weight of hyaluronic acid in the form of sodium hyaluronate (HA 1%),

in comparison with the control (synthetic skins treated with the same amount of water with no active agent: dry skin).

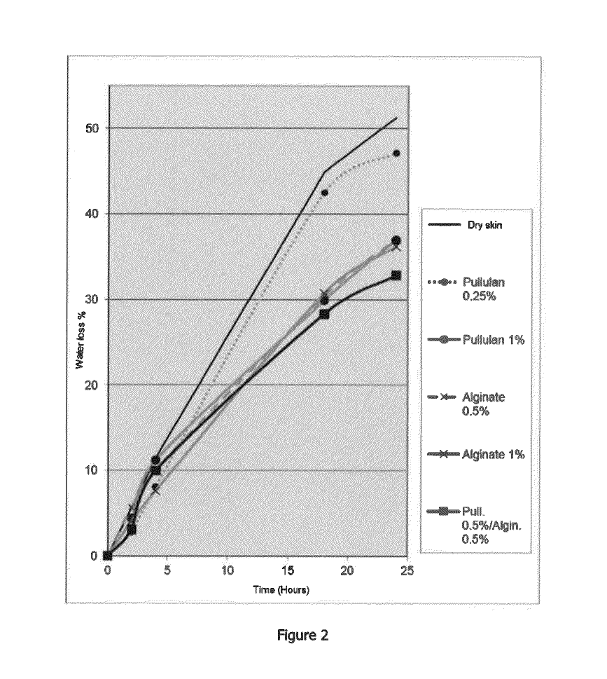

FIG. 2 represents the comparison between the measurement of the insensible losses as a function of time obtained with a composition containing the combination according to the invention (0.5% by weight of pullulan and 0.5% by weight of alginic acid in the form of sodium alginate: Pull. 0.5%/Algin. 0.5%) and the measurement of the insensible losses as a function of time obtained with:

a composition containing 0.25% by weight of pullulan (Pullulan 0.25%),

a composition containing 1% by weight of pullulan (Pullulan 1%),

a composition containing 0.5% by weight of alginic acid in the form of sodium alginate (Alginate 0.5%), and

a composition containing 1% by weight of alginic acid in the form of sodium alginate (Alginate 1%),

in comparison with the control (synthetic skins treated with the same amount of water with no active agent: dry skin).

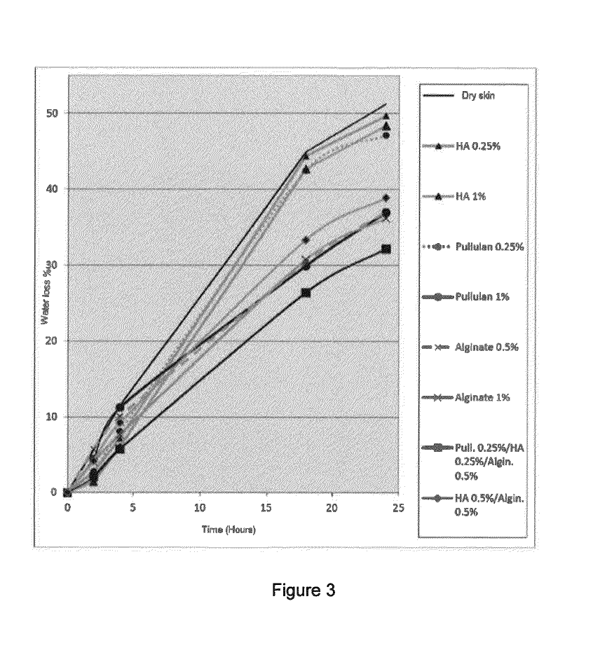

FIG. 3 represents the comparison between the measurement of the insensible losses as a function of time obtained with a composition containing the combination according to the invention (0.25% by weight of pullulan, 0.25% by weight of hyaluronic acid in the form of sodium hyaluronate and 0.5% by weight of alginic acid in the form of sodium alginate: Pull. 0.25%/HA 0.25%/Algin. 0.5%) and between the measurement of the insensible losses as a function of time obtained with:

a composition containing 0.25% by weight of pullulan (Pullulan 0.25%),

a composition containing 1% by weight of pullulan (Pullulan 1%),

a composition containing 0.25% by weight of hyaluronic acid, in the firm of sodium hyaluronate (HA 0.25%),

a composition containing 1% by weight of hyaluronic acid in the form of sodium hyaluronate (HA 1%),

a composition containing 0.5% by weight of alginic acid in the form of sodium alginate (Alginate 0.5%),

a composition containing 1% by weight of alginic acid in the form of sodium alginate (Alginate 1%), and

a composition containing 0.5% by weight of hyaluronic acid in the form of sodium hyaluronate and 0.5% by weight of alginic acid in the form of sodium alginate (HA 0.5%/Algin. 0.5%),

in comparison with the control (synthetic skins treated with the same amount of water with no active agent: dry skin).

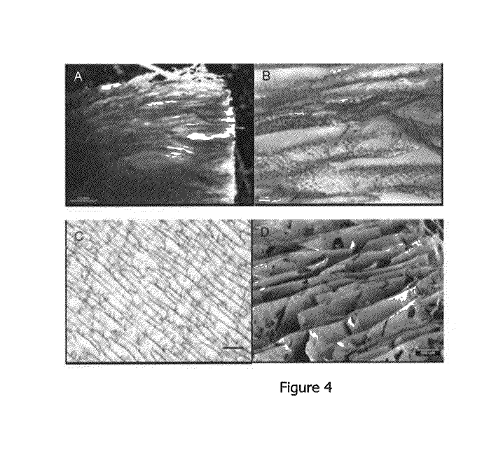

FIG. 4 represents the visualization by microscopy of the network of polymers formed by the combination according to the invention during the experiment described according to Example 6:

A. Observation of the network formed by the combination of the 3 compounds pullulan, sodium hyaluronate and sodium alginate by macrofluorescence.

B. Observation according to A at larger scale.

C. Observation of the network by optical microscopy (×10).

D. Observation of the network by transmission electron microscopy (×60).

Other objectives, features and advantages of the invention will become clear to a person skilled in the art on reading the explanatory description which refers to examples that are given solely by way of illustration and which could not in any way limit the scope of the invention.

The examples are an integral part of the present invention and any feature that appears novel with respect to any prior art from the description taken in its entirety, including the examples, is an integral part of the invention in its function and in its generality.

Thus, each example has a general scope.

Moreover, in the examples and unless otherwise indicated the temperature is expressed in degrees Celsius and the pressure is atmospheric pressure.

### Example 1

### Demonstration of the Synergistic Effect on the Reduction of the Water Losses of the Combination According to the Present Invention Containing Pullulan and Hyaluronic Acid. (FIG. 1)

### Principle:

The test used is an in vitro test measuring the amount of water evaporated as a function of time. It draws inspiration from the conventional tests in an open cylinder but was carried out after drying in an oven and not in a humid atmosphere, which increases the insensible water losses and mimics them under much harsher conditions than the conventional tests.

### Protocol:

The protocol was the following:
The various compositions to be tested were prepared by mixing, under planetary stirring, of the components at various concentrations in deionized water:
   a composition containing 0.25% by weight of pullulan (Pullulan 0.25%),
   a composition containing 1% by weight of pullulan (Pullulan 1. %),
   a composition containing 0.25% by weight of hyaluronic acid, in the form of sodium hyaluronate (HA 0.25%),
   a composition containing 1% by weight of hyaluronic acid in the form of sodium hyaluronate (HA 1%), and
   a composition containing the combination according to the invention (0.5% by weight of pullulan and 0.5% by weight of hyaluronic acid in the form of sodium hyaluronate: HA 0.5%/Pull. 0.5%).
Each composition was applied as a thin layer of 200 μm on synthetic skin (Vitroskin®). As a negative control, a synthetic skin (Vitroskin®), was treated in the same way with a composition containing only this amount of water.
The synthetic skin treated was dried in a ventilated oven at 45° C. for 1 hour.
Filling the cylindrical-shaped cells having a radius of 1 cm and a height of 2 cm with of the deionized water (around 6 cm³).
Placing the treated skin over a cell so that evaporation surface area measured is around 3.14 cm².
Depositing the cell in a ventilated oven at 45° C.
Weighing the cell and measuring the water loss at t=0, 2 hours, 4 hours, 18 hours and 24 hours.

Each composition was tested 3 times and the standard deviation was calculated.

### Results:

The compositions and the results obtained are brought together in Table 1 below:

TABLE 1
Insensible water losses measured (in % of water evaporated)

| | | Time (in hours) | | | | |
|---|---|---|---|---|---|---|
| Composition | | 0 | 2 | 4 | 18 | 24 |
| with no active | Average | 0 | 5.62 | 11.45 | 44.91 | 51.28 |
| agent | Standard deviation | 0 | 1.43 | 1.97 | 8.32 | 8.56 |
| HA 0.25% | Average | 0 | 2.73 | 7.13 | 44.35 | 49.72 |
| | Standard deviation | 0 | 0.35 | 1.01 | 9.74 | 10.53 |
| HA 1% | Average | 0 | 1.42 | 6.02 | 42.68 | 48.41 |
| | Standard deviation | 0 | 0.20 | 0.37 | 1.89 | 2.43 |
| Pullulan 0.25% | Average | 0 | 3.26 | 8.34 | 42.90 | 47.22 |
| | Standard deviation | 0 | 0.74 | 0.49 | 4.06 | 5.75 |
| Pullulan 1% | Average | 0 | 5.35 | 10.13 | 33.50 | 38.52 |
| | Standard deviation | 0 | 0.74 | 0.25 | 0.54 | 0.85 |
| HA 0.5%/ | Average | 0 | 3.40 | 6.53 | 28.34 | 35.19 |
| Pull. 0.5% | Standard deviation | 0 | 1.46 | 2.74 | 4.60 | 4.15 |

### Conclusions:

As can clearly be seen in FIG. 1, the insensible water losses are lower with a composition containing the combination according to the present invention at a content of 1% by weight (0.5% of sodium hyaluronate and 0.5% of pullulan) than for a composition containing 1% by weight of sodium hyaluronate or 1% by weight of pullulan. There is therefore clearly synergy between the components. This synergy is particularly highlighted 5 hours after application, and is very significant 10 hours after application of the composition and even stronger 18 hours after application of the composition and lasts at least 24 hours.

### Example 2

### Demonstration of the Synergistic Effect on the Reduction of the Water Losses of the Combination According to the Present Invention Containing Pullulan and Alginic Acid. (FIG. 2)

### Principle and Protocol:

The protocol indicated in Example 1 is used in the same way with the following aqueous compositions to be tested:
a composition containing 0.25% by weight of pullulan (Pullulan 0/5%),
a composition containing 1% by weight of pullulan (Pullulan 1%),
a composition containing 0.5% by weight of alginic acid in the form of sodium alginate (Alginate 0.5%),
a composition containing 1% by weight of alginic acid in the form of sodium alginate (Alginate 1%),
a composition containing the combination according to the invention containing 0.5% by weight of pullulan and 0.5% by weight of alginic acid in the form of sodium alginate: Pull. 0.5%/Algin, 0.5%, and
a composition constituted of water with no active agent.

### Results:

The compositions and the results obtained are brought together in Table 2 below:

TABLE 2
Insensible water losses measured (in % of water evaporated)

| | | Time (in hours) | | | | |
|---|---|---|---|---|---|---|
| Composition | | 0 | 2 | 4 | 18 | 24 |
| with no active | Average | 0 | 5.62 | 11.45 | 44.91 | 51.28 |
| agent | Standard deviation | 0 | 1.43 | 1.97 | 8.32 | 8.56 |
| Alginate 0.5% | Average | 0 | 5.70 | 10.04 | 30.37 | 37.04 |
| | Standard deviation | 0 | 0.25 | 0.47 | 3.50 | 4.87 |
| Alginate 1% | Average | 0 | 4.08 | 7.64 | 30.71 | 36.20 |
| | Standard deviation | 0 | 1.05 | 1.61 | 3.10 | 2.25 |
| Pullulan 0.25% | Average | 0 | 3.26 | 8.34 | 42.90 | 47.22 |
| | Standard deviation | 0 | 0.74 | 0.49 | 4.06 | 5.75 |
| Pullulan 1% | Average | 0 | 5.35 | 10.13 | 33.50 | 38.52 |
| | Standard deviation | 0 | 0.74 | 0.25 | 0.54 | 0.85 |
| Pull. 0.5%/ | Average | 0 | 3.10 | 9.98 | 28.30 | 32.84 |
| Algin. 0.5% | Standard deviation | 0 | 0.79 | 1.07 | 2.86 | 2.66 |

### Conclusions:

As can clearly be seen in FIG. 2, the insensible water losses are lower for a composition containing the combination according to the present invention at a content of 1% by weight (0.5% of sodium alginate and 0.5% of pullulan) than for a composition containing 1% by weight of sodium alginate or 1% by weight of pullulan. There is therefore clearly synergy between the components. This synergy is particularly highlighted 12 hours after application, and is very significant 15 hours after application of the composition and even stronger 18 hours after application of the composition and lasts at least 24 hours.

### Example 3

### Demonstration of the Synergistic Effect n the Reduction of the Water Losses of the Combination According to the Present Invention Containing Pullulan, Hyaluronic Acid and Alginic Acid. (FIG. 3)

### Principle and Protocol:

The protocol indicated in Example 1 is used in the same way with the following aqueous compositions to be tested:
a composition containing 0.25% by weight of pullulan (Pullulan 0.25%),
a composition containing 1% by weight of pullulan (Pullulan 1%),
a composition containing 0.25% by weight of hyaluronic acid, in the form of sodium hyaluronate (HA 0.25%),
a composition containing 1% by weight of hyaluronic acid in the form of sodium hyaluronate (HA 1%),
a composition containing 0.5% by weight of alginic acid in the form of sodium alginate (Alginate 0.5%),
a composition containing 1% by weight of alginic acid in the form of sodium alginate (Alginate 1%),
a composition containing 0.5% by weight of hyaluronic acid in the form of sodium hyaluronate and 0.5% by weight of alginic acid in the form of sodium alginate (HA 0.5%/Algin.0.5%),
a composition containing 0.25% by weight of pullulan, 0.25% by weight of hyaluronic acid in the form of sodium hyaluronate and 0.5% by weight of alginic acid in the form of sodium alginate: Pull. 0.25%/HA 0.25%/Algin. 0.5%, and
a composition constituted of water with no active agent

### Results:

The compositions and the results obtained are brought together in Table 3 below:

TABLE 3
Insensible water losses measured (in % of water evaporated)

| | | Time (in hours) | | | | |
|---|---|---|---|---|---|---|
| Composition | | 0 | 2 | 4 | 18 | 24 |
| with no active | Average | 0 | 5.62 | 11.45 | 44.91 | 51.28 |
| agent | Standard deviation | 0 | 1.43 | 1.97 | 8.32 | 8.56 |
| HA 0.25% | Average | 0 | 2.73 | 7.13 | 44.35 | 49.72 |
| | Standard deviation | 0 | 0.35 | 1.01 | 9.74 | 10.53 |
| HA 1% | Average | 0 | 1.42 | 6.02 | 42.68 | 48.41 |
| | Standard deviation | 0 | 0.20 | 0.37 | 1.89 | 2.43 |
| Alginate 0.5% | Average | 0 | 5.70 | 10.04 | 30.37 | 37.04 |
| | Standard deviation | 0 | 0.25 | 0.47 | 3.50 | 4.87 |
| Alginate 1% | Average | 0 | 4.08 | 7.64 | 30.71 | 36.20 |
| | Standard deviation | 0 | 1.05 | 1.61 | 3.10 | 2.25 |
| Pullulan 0.25% | Average | 0 | 3.26 | 8.34 | 42.90 | 47.22 |
| | Standard deviation | 0 | 0.74 | 0.49 | 4.06 | 5.75 |
| Pullulan 1% | Average | 0 | 5.35 | 10.13 | 33.50 | 38.52 |
| | Standard deviation | 0 | 0.74 | 0.25 | 0.54 | 0.85 |
| HA 0.5%/ | Average | 0 | 4.17 | 9.21 | 33.32 | 38.90 |
| Algin. 0.5% | Standard deviation | 0 | 0.87 | 1.80 | 1.18 | 1.44 |
| Pull. 0.25%/ | Average | 0 | 2.05 | 5.73 | 26.36 | 32.17 |
| HA 0.25%/ | Standard deviation | 0 | 0.55 | 1.43 | 2.53 | 2.02 |
| Algin. 0.5% | | | | | | |

### Conclusions:

As can clearly be seen in FIG. 3, the insensible water losses are more reduced for a composition containing the combination according to the present invention at a content of 1% by weight (0.5% of alginic acid, 0.25% of hyaluronic acid and 0.5% of pullulan) than for a composition containing 1% by weight of alginic acid or 1% by weight of pullulan or 1% by weight of hyaluronic acid or even 0.5% by weight of hyaluronic acid and 0.5% by weight of alginic acid. There is therefore clearly synergy between the 3 components. This synergy is particularly highlighted 4 hours after application of the composition and lasts at least 24 hours.

This synergy is greater than for the simple combination of hyaluronic acid and pullulan as exemplified in Example 1 or that of alginic acid and pullulan as exemplified in Example 2.

Furthermore, the combination of alginic acid and of hyaluronic acid has no synergistic effect on the insensible water losses.

### Example 4

### Evaluation of the Properties of a Composition According to the Invention Containing the Combination of Sodium Alginate, Sodium Hyaluronate and Pullulan on the Water Content

### Principle:

This study consists in evaluating ex viva, by measuring the dielectric conductivity, the horny layer moisturizing properties of the combination according to the invention in comparison with compositions that do not comprise this combination.

This technique is based on the fact that the drier the horny layer, the lower its electric conduction, this due to the bipolarity of the water molecules and to the electric field that they thus induce in the horny layer. This technique is conventionally used for measuring the moisturizing power of topical active agents and was described in the following publications: OBATA M, TAGAMI H: A rapid in vitro test to assess skin moisturizers, J. Soc. Cosmet. Chem., vol. 41, 235-242, 1990; OBATA M, TAGAMI H: Electrical determination of water content and concentration profile in simulation model of in vivo stratum corneum, J. Invest. Dermatol., vol. 92, 854-859, 1989.

### Protocol:

Biopsies of normal human abdominal skin resulting from surgical waste are removed and the epidermal layers are separated from the dermis after heating at 60° C. for two minutes. The horny layer “stratum corneum” is isolated by enzymatic digestion according to the protocol described in the publication ROCHEFORT A, DROUOT P, LEDUC M, VASSALET R, AGACHE P: A new technique for evaluation of cosmetics effect on mechanical properties of stratum corneum and epidermis in vitro. Int J. Cosm. Sci., vol. 8, 27-36, 1986 with the adaptations mentioned below.

The model of horny layer is placed in chambers of defined relative humidity: 44%-saturated with a potassium carbonate solution for 1 h.

Each model of horny layer is tested under the following conditions:
stratum corneum with no treatment, (TN1)
stratum corneum treated with a placebo hydrogel (TN2) for which composition A is the following:

| Trade name | INCI | Amount (%) |
|---|---|---|
| water | Water | qs for 100% |
| Elestab ™ 50J | Chlorphenesin (and) | 0.35 |
| | methylparaben | |
| Carbopol 980 | Carbomer | 0.60 |
| Water | Water | 29.40 |
| 16% NaOH | Sodium hydroxide | 0.75 |

stratum corneum treated with the hydrogel according to composition A containing 1% of composition B below, and then known as TP1, or containing 3% of composition B, then known as TP2:
   composition B:

| Name | Amount (%) |
|---|---|
| water | 55.58 |
| Pentylene glycol | 2 |
| Potassium phosphate | 0.02 |
| Trehalose | 10 |
| Serine | 3 |
| Caprylyl glycol | 0.5 |
| Glycerin | 17.5 |
| Glyceryl polyacrylate | 0.3 |
| Urea | 10 |
| Disodium phosphate | 0.1 |

stratum corneum treated with a hydrogel of composition A containing 1% (M1) or 2% (M2) or 3% (M3) of a mixture of cosmetic ingredients according to Example 7 described below (alginate/HA/pullulan ratio: 21/1).

The models are then treated with the topical preparations by three consecutive applications spaced 30 minutes apart. The doses applied are 1 mg/cm².

The measurements are repeated 12 times for each sample and for each time interval.

The dielectric conductivity was measured before treatment, then at 30 minutes, 1, 4, 6, 24 and 48 hours after the treatment.

### Results:

The results are presented in Table 41 The results presented in Table 4.2 are expressed as a percentage increase of the dielectric conductivity after treatment with the compositions mentioned containing the products tested relative to the dielectric conductivity measurement obtained with the horny layer treated with the so-called placebo composition (negative control 2: TN2).

TABLE 4
Measurements of the dielectric conductivity after
application of the compositions to be tested

| | | Time in hours | | | | | | | | |
|---|---|---|---|---|---|---|---|---|---|---|
| Composition tested | | 0 | 0.5 | 1 | 2 | 4 | 6 | 24 | 30 | 48 |
| TN1 | Av | 14.3 | 15.0 | 15.6 | 15.3 | 16.1 | 17.1 | 15.8 | 15.6 | 16.0 |
| | SD | 0.8 | 0.9 | 0.8 | 0.9 | 1.3 | 1.2 | 1.0 | 1.0 | 1.3 |
| TN2 Placebo | Av | 13.0 | 34.4 | 25.3 | 21.3 | 20.0 | 18.6 | 18.4 | 17.4 | 18.5 |
| | SD | 0.8 | 2.0 | 1.4 | 1.8 | 1.8 | 1.7 | 1.5 | 1.3 | 1.2 |
| TP1 | Av | 15.1 | 39.0 | 28.4 | 31.3 | 27.2 | 25.0 | 25.0 | 24.3 | 26.4 |
| | SD | 1.0 | 1.7 | 1.1 | 1.9 | 1.3 | 1.3 | 1.2 | 1.5 | 1.2 |
| TP2 | Av | 15.3 | 48.5 | 35.6 | 31.5 | 31.8 | 30.9 | 28.4 | 29.2 | 30.2 |
| | SD | 0.9 | 2.7 | 2.2 | 2.6 | 2.0 | 1.8 | 1.5 | 2.2 | 2.6 |
| M1 1% | Av | 14.5 | 46.3 | 33.2 | 33.1 | 31.6 | 31.9 | 30.6 | 27.7 | 25.6 |
| HA/Algin/pull | SD | 0.6 | 2.5 | 1.5 | 1.2 | 2.5 | 2.0 | 2.1 | 1.7 | 2.2 |
| M2 2% | Av | 15.2 | 54.7 | 41.2 | 38.7 | 38.9 | 37.0 | 33.8 | 32.7 | 33.6 |
| HA/Algin/pull | SD | 0.6 | 2.5 | 1.4 | 1.6 | 1.1 | 1.3 | 1.2 | 1.0 | 2.6 |
| M3 3% | Av | 15.9 | 67.3 | 53.2 | 47.6 | 45.8 | 44.1 | 39.4 | 40.6 | 38.0 |
| HA/Algin/pull | SD | 0.9 | 3.4 | 2.8 | 3.1 | 3.2 | 2.6 | 2.4 | 2.2 | 2.9 |

Av denotes the average;
SD denotes standard deviation

TABLE 4.2
Percentage variation relative to the TN2 placebo -
results in % as a function of time

| | Time in hours | | | | | | | | |
|---|---|---|---|---|---|---|---|---|---|
| Composition tested | 0 | 0.5 | 1 | 2 | 4 | 6 | 24 | 30 | 48 |
| TP2: placebo + 3% | 17.3 | 41.0 | 41.0 | 48.2 | 59.2 | 66.1 | 54.1 | 67.8 | 63.5 |
| hydrating complex | | | | | | | | | |
| M3: placebo + 3% | 22.0 | 95.5 | 110.5 | 124.2 | 129.2 | 136.7 | 113.7 | 133.3 | 105.9 |
| (hydrating complex + | | | | | | | | | |
| Mixture HA/Pu/ALG | | | | | | | | | |
| (1/1/2)) | | | | | | | | | |

### Conclusions:

The water content was evaluated by measuring the dielectric conductivity, which is proportional to the state of hydration of the horny layer.

The untreated negative control demonstrates that the water content in the biopsy and under the test conditions was stable over time. The application of the compositions gave rise to an increase of the dielectric conductivity and therefore of the water content of the horny layer.

The compositions M1, M2 and M3 containing the combination according to the invention demonstrate an ability to increase the water content of the skin and makes it possible to conclude that they have a significant moisturizing effect versus the placebo composition TN.

The comparison of the composition M1 containing the combination according to the invention with the composition TP1, and of compositions M2 and M3 containing the combination according to the invention with the compositions TP1 and TP2 demonstrates that the combination according to the invention has a much higher efficacy, at all the time intervals up to 48 hours, which translates into an immediate and long-lasting effect.

This example demonstrates that the combination according to the invention very effectively and long-lastingly increases the water content of the skin and/or mucous membranes and therefore constitutes a very good moisturizing agent.

### Example 5

### Evaluation of the Content of Active Ingredients with a Composition Containing the Combination According to the Invention

### Principle:

This ex vivo study is carried out on Franz cells making it possible to study the diffusion of the compositions tested through a biopsy of skin as a function of time.

### Protocol:

Complete biopsies of normal human abdominal skin were obtained from surgical waste and inserted at the interface of the donor and receptor compartments of the Franz cells. The compositions tested were applied to the so-called donor/upper face of the cells. PBS buffer was used as receptor fluid.

The compositions tested were the following:
Positive control: composition B (containing serine and urea respectively at 3% and 10%) (Control composition)
Mixture of cosmetic ingredients according to Example 7 below (Composition according to the invention)

The Franz cells containing the biopsies were incubated for 48 hours at 37° C. with the compositions to be tested. The compositions to tested were thus applied in the amount of 300 microliters to the upper/donor part of the cells, which corresponds to the application of 9 mg of serine and 30 mg of urea for all of the compositions. The biopsies were recovered after 6, 10, 24 and 48 hours of treatment and the horny layer, stratum corneum, was removed by stripping using eight adhesive tapes (tape-stripping technique—D-Squame™, CuDerm Corp.). The 8 adhesive tapes were reunited to reconstitute the horny layer and the serine and the urea contained were extracted by PBS buffer and quantified by high-performance liquid chromatography (HPLC) and quantified by a conventional standard. The value obtained with the PBS was subtracted from each measurement. The experiment was carried out in quintuplicate (n=5).

### Results:

The significance was determined by a Student's t-test with a threshold set at 5% (p<0.05)

The results for serine are presented in Table 5.1 and for urea in Table 5.2.

TABLE 5.1
Storage of serine in the stratum corneum

| Compositions | | Time in hours | | | |
|---|---|---|---|---|---|
| tested | | 6 | 10 | 24 | 48 |
| Control | Average | 4.53 | 6.30 | 18.66 | 21.34 |
| composition | Standard | 3.99 | 4.23 | 11.35 | 5.35 |
| | deviation | | | | |
| Composition | Average | 21.54 | 69.78 | 79.17 | 81.57 |
| according to | Standard | 4.75 | 16.89 | 59.68 | 10.51 |
| invention | deviation | | | | |

| | | | | |
|---|---|---|---|---|
| Comparison “composition | +353 | +313 | +324 | +282 |
| according to invention vs | | | | |
| control composition (in | | | | |
| %)” | | | | |

TABLE 5.2
Storage of urea in the stratum corneum

| Compositions | | Time in hours | | | |
|---|---|---|---|---|---|
| tested | | 6 | 10 | 24 | 48 |
| Control | Average | 49.69 | 46.40 | 89.82 | 104.11 |
| composition | Standard | 10.79 | 10.92 | 35.05 | 34.14 |
| | deviation | | | | |
| Composition | Average | 125.12 | 228.41 | 269.02 | 272.74 |
| according to | Standard | 29.74 | 56.65 | 130.33 | 92.87 |
| invention | deviation | | | | |

| | | | | |
|---|---|---|---|---|
| Comparison “composition | +152 | +392 | +200 | +162 |
| according to invention vs | | | | |
| control composition (in | | | | |
| %)” | | | | |

### Conclusions:

The results demonstrate that the serine contained in the composition according to the invention is much better retained in the horny layer after 6 hours but also after 10, 24 and 48 hours than when it is in a control composition with no polymer.

Similarly, the urea, when applied with the composition according to the invention is better retained and kept in the horny layer at 6 hours but also after 10, 24 and 48 hours.

The results of this ex vivo study demonstrate that, when they are applied in the form of a composition according to the invention, serine and urea are better retained in the horny layer and their content therein is increased.

The combination according to the invention therefore increases the content of active agents in the stratum corneum.

### Example 6

### Visualization by Microscopy of the Network of Polymers Formed by the Combination According to the Invention

### Protocol:

An aqueous solution containing 0.25% pullulan, 0.25% sodium hyaluronate and 0.5% sodium alginate by weight, in a 1/1/2 ratio is homogenized then freeze-dried. The combination thus obtained in the form of a dehydrated network is then finely cut (5×10 cm) for observation by macrofluorescence or by transmission electron microscopy.

For observation by optical microscopy, 1 ml of the aqueous solution containing 0.25% pullulan, 0.25% sodium hyaluronate and 0.5% sodium alginate by weight, in a 1/1/2 ratio is withdrawn before freeze-drying then left to dry for 16 hours at room temperature (20° C.) before observation.

### Results:

The results are presented in FIG. 4.

### Conclusions:

In the 1/1/2 ratio, the combination according to the invention of pullulan, hyaluronic acid and alginic acid forms a multilayered molecular network as observed above by microscopy. In particular, the observation by transmission electron microscopy (I)) clearly shows the sheet structure of the network formed. The formation of this molecular network which induces a retarding effect on the percutaneous diffusion of the active ingredient(s) contained in the compositions according to the invention and thus increases and prolongs over time the content of active ingredient(s), as demonstrated in Example 5 above.

### Example 7

### Mixture of Cosmetic or Pharmaceutical Ingredients According to the Invention containing the Combination of Sodium Alginate, Sodium Hyaluronate and Pullulan

A mixture of cosmetic or pharmaceutical ingredients having the formulation below in percentages by weight is prepared.

| Name | Amount in % |
|---|---|
| Sodium Alginate | 0.5 |
| Pentylene glycol | 2 |
| Potassium phosphate | 0.02 |
| Trehalose | 10 |
| Serine | 3 |
| Sodium hyaluronate | 0.25 |
| Caprylyl glycol | 0.5 |
| Pullulan | 0.25 |
| Glycerin | 17.5 |
| Glyceryl polyacrylate | 0.3 |
| Urea | 10 |
| Disodium phosphate | 0.1 |
| Water | qs |

The preparation process is the following: in water buffered to pH 6 with a mixture of disodium phosphate/potassium phosphate salts, the sodium hyaluronate and pullulan are added. Once the mixture of polymers is obtained, the urea, trehalose, serine and glycerine are added. After complete solubilization, the sodium alginate, pentylene glycol, caprylyl glycol and the gel based on glyceryl polyacrylate are added so as to stabilize the product.

### Example 8

### Composition According to the Invention in the Form of a Moisturizing Lotion for the Body and/or the Face

| | | Amount (% of total |
|---|---|---|
| Phase | Name | weight) |
| A | Water | 69.90 |
| A | Disodium EDTA | 0.05 |
| A | Xanthan gum | 0.20 |
| B | Steareth-2 | 2.00 |
| B | Steareth-21 | 2.50 |
| B | Cetearyl alcohol | 1.00 |
| B | Propylheptyl caprylate | 15.00 |
| C | Mixture of cosmetic ingredients according | 3.00 |
| | to Example 7 comprising the combination | |
| | according to the invention | |
| D | Water | 1.00 |
| D | Sodium hydroxide (30% in solution) | 0.10 |
| E | Mixture of phenoxyethanol, chlorphenesin, | 1.25 |
| | benzoic acid, butylene glycol, sorbic acid | |
| | (Germazide ™ PBS) | |
| F | Mixture of polyacrylate-X, isohexadecane | 4.00 |
| | and polysorbate 60 (Sepigel ™ SMS 60) | |

The lotion is prepared by the standard methods in the field, well known to those skilled in the art, by mixing the 6 phases.

### Example 9

### In Vivo Evaluation of the Moisturizing Effect of a Composition According to the Invention in Cosmetic or Pharmaceutical Form

### Principle:

This study makes it possible to determine in vivo the immediate hydration provided by the combination according to the invention using a corneometer after a single application.

### Protocol:

The study was conducted on 22 women aged from 18 to 65 years old.

A slight destructuring of the epidermis in the test region was carried out before the test, consisting of a pretreatment of 3 days of cleansing with soap, twice a day to mimic “very dry” skin. The composition according to Example 8 without the mixture of cosmetic ingredients according to the invention, referred to as the placebo composition, or with the mixture of cosmetic ingredients according to the invention, referred to as the composition according to the invention, was applied to the forearm as a single application (approximate amount of 40 microliters over 3×3 cm²).

The hydration was measured at 30 minutes and 4, 8, 24 and 48 hours using a corneometer after application and in triplicate.

### Results:

The results are expressed as the percentage variation of the value measured relative to the initial value measured before the test, which translates into the percentage improvement of the hydration.

TABLE 8

| | Time after application (hours) | | | | |
|---|---|---|---|---|---|
| Composition tested | 0.5 | 4 | 8 | 24 | 48 |
| Placebo composition | 58 | 14 | 20 | 24 | 28 |
| Composition | 73 | 24 | 30 | 32 | 33 |
| according to the | | | | | |
| invention | | | | | |

The difference observed between the placebo composition and the composition according to the invention is always significant according to the t-test (p<0.05)

### Conclusion

The composition according to the invention improved the water content of the skin significantly compared to the placebo composition. This improvement is immediate, from 30 minutes onwards and long-lasting over time.

The experiment was repeated on half the face, consisting of the application of the composition according to the invention or of the placebo composition to the face, this application being repeated twice a day for 20 days and the morning of the 21^{st} day on a population of similar women, which also showed that, on the 23^{rd} and 26^{th} day, i.e. two days or five days after stopping the applications, the water content of the skin of the women who used the placebo composition, measured with the corneometer, had returned to its initial state whereas the face of the women who used the composition according to the invention still had a significant improvement of the water content of their skin even after stopping the treatment (+14% on the 23^{rd} day, i.e. 2 days after stopping the treatment and +12% on the 26^{th} day) which conveys the persistence of this effect.

1-29. (canceled)

30. A combination of pullulan or of a derivative thereof with a mixture of polysaccharides consisting of hyaluronic acid or a salt or derivative thereof and alginic acid or a salt or derivative thereof, the weight ratio of pullulan or derivatives/hyaluronic acid, salts or derivatives/alginic acid, salts or derivatives being in the range 1/0.001/0.001 to 1/100/100.

31. The combination according to claim 30, wherein the pullulan has a molecular weight of less than 500 kDa.

32. The combination according to claim 30, wherein the hyaluronic acid, the salts or esterified derivatives thereof have a molecular weight of greater than 20 kDa.

33. The combination according to claim 30, wherein the derivatives of hyaluronic acid are the esterified derivatives or the organomineral silicon-based derivatives or mixtures thereof.

34. The combination according to claim 30, wherein the combination comprises a salt of hyaluronic acid.

35. The combination according to claim 30, wherein the combination comprises a salt of alginic acid.

36. The combination according to claim 30, wherein the derivatives of alginic acid are esterified derivatives or organomineral silicon-based derivatives or mixtures thereof.

37. The combination according to claim 30 wherein the weight ratio of pullulan, or derivatives and the mixture of polysaccharides is in the range 1/0.002 to 1/200.

38. The combination according to claim 30 wherein the weight ratio of pullulan, or derivatives/hyaluronic acid, salts or derivatives/alginic acid, salts or derivatives is in the range 1/1/1 to 1/10/10.

39. The combination according to claim 30, wherein the weight ratio of pullulan, or derivatives/alginic acid, salts or derivatives is in the range 1/1 to 1/2.

40. The combination according to claim 30, wherein the weight ratio of pullulan, or derivatives/hyaluronic acid, salts or derivatives/alginic acid, salts or derivatives is 1/1/2.

41. A method comprising applying a combination of pullulan or of a derivative thereof with a polysaccharide chosen from the group consisting of hyaluronic acid, a salt or derivative thereof, alginic acid, a salt or derivative thereof, and a mixture of these polysaccharides, to skin or mucous membranes, thereby reducing the insensible water losses and/or increasing the water content of the skin and/or mucous membranes and/or increasing and/or prolonging the content of cosmetic and/or pharmaceutical active ingredients in the skin and/or the mucous membranes and/or maintaining it over time.

42. A moisturizing agent for the skin and/or mucous membranes comprising a combination of pullulan or of a derivative thereof with a polysaccharide chosen from hyaluronic acid, a salt or derivative thereof, alginic acid, a salt or derivative thereof, and a mixture of these polysaccharides.

43. The moisturizing agent according to claim 41 wherein the weight ratio of pullulan or derivatives/hyaluronic acid, salts or derivatives/alginic acid, salts or derivatives being in the range 1/0.001/0.001 to 1/100/100.

44. A mixture of cosmetic or pharmaceutical ingredients intended to be incorporated into a cosmetic or pharmaceutical composition, said mixture comprising the combination according to claim 30 and a suitable cosmetic or pharmaceutical carrier.

45. The mixture according to claim 44, wherein the mixture comprises the combination in a content of between 0.001% and 20% by weight of dry matters relative to the total weight of the mixture of ingredients.

46. The mixture according to claim 44, wherein the mixture comprises:
pullulan or a cosmetically or pharmaceutically acceptable derivative thereof in a content between 0.0001% and 10% by weight of dry matters relative to the total weight of the mixture of cosmetic or pharmaceutical ingredients; and
hyaluronic acid or a cosmetically or pharmaceutically acceptable salt or derivative thereof in a content between 0.0001% and 10% by weight of dry matters relative to the total weight of dry matters of the mixture of cosmetic or pharmaceutical ingredients; and/or
alginic acid or a cosmetically or pharmaceutically acceptable salt or derivative thereof in a content between 0.0001% and 10% by weight of dry matters relative to the total weight of the mixture of cosmetic or pharmaceutical ingredients.

47. The mixture according to claim 44, wherein the mixture additionally comprises another moisturizing agent selected from the group consisting of trehalose, serine, urea and mixtures thereof.

48. The mixture according to claim 44, wherein the mixture contains alginic acid or a cosmetically or pharmaceutically acceptable salt or derivative thereof in a content between 0.1% and 2% by weight of dry matters relative to the total weight of the mixture of cosmetic or pharmaceutical ingredients.

49. A cosmetic or pharmaceutical composition intended for topical administration comprising the combination according to claim 30 and a suitable cosmetic or pharmaceutical carrier.

50. The composition according to claim 49, wherein the composition comprises the combination in a content between 0.0001% and 20% by weight of dry matters relative to the total weight of the composition.

51. The composition according to claim 49, wherein the composition comprises:
pullulan or a cosmetically or pharmaceutically acceptable derivative thereof in a content between 0.0001% and 10% by weight of dry matters relative to the total weight of the composition; and
hyaluronic acid or a cosmetically or pharmaceutically acceptable salt or derivative thereof in a content between 0.0001% and 10% by weight of dry matters relative to the total weight of dry matters of the composition; and/or
alginic acid or a cosmetically or pharmaceutically acceptable salt or derivative thereof in a content between 0.0001% and 10% by weight of dry matters relative to the total weight of the composition.

52. The composition according to claim 49, wherein the composition contains alginic acid or a cosmetically or pharmaceutically acceptable salt or derivative thereof in a content between 0.001% and 1% by weight of dry matters relative to the total weight of the composition.

53. The composition according to claim 52, wherein the composition contains alginic acid or a cosmetically or pharmaceutically acceptable salt or derivative thereof in a content between 0.007% and 0.2% by weight of dry matters relative to the total weight of the composition.

54. The composition according to claim 49, wherein the composition is a serum, a lotion, a cream, a milk, an ointment, a paste, a foam, an emulsion, a hydrogel, a shower gel, a mask, a stick, a patch, or makeup powders.

55. The composition according to claim 49, wherein the composition contains the combination of pullulan, or derivatives/hyaluronic acid, salts or derivatives/alginic acid, salts or derivatives in a ratio in the range 1/1/1 to 1/10/10, preferably 1/1/1 to 1/2/5.

56. A method comprising applying the cosmetic composition according to claim 49 to skin and/or mucous membranes, thereby maintaining or improving the state of hydration of the skin and/or mucous membranes, and/or for preventing or slowing down the appearance of the signs of cutaneous and/or mucosal dryness, and/or for treating cutaneous and/or mucosal dryness conditions such as the squamous states and/or itching and/or tautness associated with dry skin and/or mucous membranes and/or for preventing or reducing the appearance of wrinkles linked to cutaneous dryness, and/or for improving the comfort of dry skin and/or mucous membranes, and/or for treating skin and/or mucous membranes having an appearance that is rough to look at and/or to the touch and/or for the treatment and/or the prevention of fissures.

57. The composition according to claim 49, for the use thereof in the treatment and/or the prevention of scurf or pityriasis alba and/or cracks and/or atopic dermatitis and/or ichthyosis and/or conditions of dryness of the skin or mucous membrane that accompany cutaneous and/or mucosal pathologies such as eczema.

58. A cleansing composition, in particular detergent composition, comprising the combination according to claim 30.
